(19) United States
(12) Patent Application Publication
(10) Pub. No.: US 2020/0030452 A1
(43) Pub. Date: Jan. 30, 2020

(54) Process for the Manufacture of a Tumor-Vasculature Targeting Antitumor Agent

(71) Applicant: IF7Cure, Inc., San Diego, CA (US)

(72) Inventors: Jalal Haddad, San Diego, CA (US); Michiko Fukuda, San Diego, CA (US)

(21) Appl. No.: 16/337,627

(22) PCT Filed: Oct. 2, 2017
(86) PCT No.: PCT/US2017/054801

§ 371 Date: Mar. 28, 2019

Related U.S. Application Data
(60) Provisional application No. 62/402,127, filed on Sep. 30, 2016.

(51) Int. Cl. A61K 47/64; A61K 31/4745

(52) U.S. Cl. CPC A61K 47/64; A61K 31/4745

(57) ABSTRACT

A synthetic process for manufacture of a tumor-vascular targeting antitumor agent wherein the antitumor agent comprises an annexin-1 binding peptide conjugated to an anticancer drug through a linker. An efficient, practical, reproducible and scalable process for the manufacturing of a tumor-vascular targeting antitumor agent wherein the antitumor agent comprises an annexin-1 binding peptide conjugated to an anticancer drug through a linker with high purity and yield.

### CROSS-REFERENCE TO RELATED APPLICATIONS

This application claims the benefit of priority from U.S. Provisional Patent Application No. 62/402,127 filed Sep. 30, 2016.

### FIELD OF THE INVENTION

The present invention relates to the synthetic process for manufacturing the tumor-vasculature targeting antitumor agent, IF7-SN38 via the key intermediate, BCH-SN38. IF7-SN38 is an antitumor agent comprising the annexin 1-binding peptide, designated as IF7, which is conjugated to potent anticancer drug SN38 through a linker, designated as BCH.

### BACKGROUND OF THE INVENTION

A major hurdle in synthesizing a drug candidate on a manufacturing scale is the lack of an efficient process that could lead to formation of the intermediates and product in high yield and purity economically. The early medicinal chemistry protocols for the syntheses of active pharmaceutical ingredients (APIs) require major process development studies and optimization to transform the mg-scale protocols to processes amenable for the large-scale manufacturing of APIs.

The synthesis of IF7-SN38 on milligram scale was reported by Hatakeyama et al., and the same author, Michiko Fukuda, (Proc. Natl. Acad. Sci. USA. 2011 Dec. 6; 108(49): 19587-19592; WO2011079304 A1) according to the method described by Meyer-Losic et al., Clin. Cancer Res. 2008; 14: 2145-53, with some modifications. However, the synthesis had serious problems regarding scalability and reproducibility generating side reactions, which in turn resulting in tedious purifications, poor yields and purities. Due to the formation of many byproducts in the reaction mixtures, several reverse phase purifications were required to purify the penultimate BCH-SN38 and final product. As a result of a lengthy purifications processes, degradations and formation of new byproducts were observed during purification processes, which made the synthetic process cumbersome, irreproducible and impractical for large scale production of IF7-SN38.

Accordingly, the objective of the present invention is to develop an efficient, practical, reproducible and scalable process for the manufacturing of cGMP grade IF7-SN38 with high purity and yield.

In light of the above, it is an object of the present invention to provide the desired features described herein.

### SUMMARY OF THE INVENTION

Disclosed are compositions comprising a moiety and a peptide, the peptide comprising an amino acid sequence that can bind to a carbohydrate receptor on a cell. The compositions can be used for the treatment of various types of diseases, including cancers. The carbohydrate receptor can be annexin 1. The amino acid sequence can selectively bind the carbohydrate receptor. The subject can comprise a cell. The cell can be an endothelial cell. The peptide can be an annexin 1-binding compound. The amino acid sequence can be an annexin 1-binding compound.

The peptide can comprise at least 6 amino acids. The peptide can comprise at least 7 amino acids. The peptide can comprise at least 8 amino acids. The peptide can comprise at least 9 amino acids. The peptide can further comprise a moiety peptide. The peptide can be head to tail circular.

The compositions disclosed herein can comprise one or more moieies. For example, moieties can be molecules, conjugates, associations, compositions, and mixture. The moiety can be a small molecule, pharmaceutical drug, toxin, fatty acid, detectable marker, conjugating tag, nanoshell, or enzyme. Example moieties include, but are not limited to, anti-angiogenic agents, pro-angiogenic agents, cancer chemotherapeutic agents, cytotoxic agents, anti-inflammatory agents, anti-arthritic agents, polypeptides, nucleic acid molecules, small molecules, nanoparticles, and microparticles. At least one of the moieties can be a therapeutic agent. Examples of therapeutic agents can be paclitaxel and docetaxel.

The composition can further comprise a linker connecting the moiety and the peptide. The composition can further comprise a pharmaceutically acceptable carrier. The composition can further comprise a detectable agent. The composition can further comprise a therapeutic agent. The composition can further comprise an anti-cancer agent. The composition can further comprise a plurality of peptides, wherein at least one of the peptides comprises an amino acid sequence that selectively binds to tumor vasculature.

The moiety can be covalantely linked to the peptide. The moiety can be linked to the amino terminal end of the peptide. The moiety can be linked to the carboxy terminal end of the peptide. The moiety can be linked to an amino acid within the peptide. The moiety can be a camptothecin (CPT) derivative. The moiety can be SN38. The moiety can comprise a detectable agent. The moiety can comprise a therapeutic agent. The therapeutic agent can comprise a compound or composition for treating cancer. The therapeutic agent can comprise a compound or composition to induce programmed cell death or apoptosis. The therapeutic agent can be Abraxane. The therapeutic agent can be paclitaxel. The therapeutic agent can be docetaxel. At least one of the moieties can be a detectable agent. The detectable agent can be FAM.

The disclosed annexin 1-biding compounds and moieties can be linked in any useful way. For example, annexin 1-binding compounds and moeities can be covalently coupled (directly or indirectly), noncovalently coupled (directly or indirectly), or both. Direct coupling can be via a covalent bond between the annexin 1-binding compound and the moiety. The covalent bond in such cases can be considered the linkage between the annexin 1-binding compound and the moiety. Indirect coupling can be via one or more intervening molecules or components. Usefule direct coupling can be via a linker. The linker, any bond in the linker that couples the annexin 1-binding compound and the moiety, the bond between the annexin 1-binding compound and the linker, and/or the bond between the moiety and the linker can be considered a linkage. Any suitable linker can be used. For example, the linker can be an oligomer, such as a peptide or peptide mimetic.

The compositions disclosed here can be prepared and/or administered as a pharmaceutically acceptable inorganic or organic salt, formed by reaction with inorganic or organic acids (P. H. Stahl and C. G. Wermuth, editors, Handbook of Pharmaceutical Salts: Properties, Selection and Use, Weinheim/Zürich: Wiley-VCH/VHCA, 2002). Exemplary examples of inorganic and organic acids include, but are not limited to, hydrochloric acid, hydrobromic acid, hydrofluoric acid, boric acid, perchloric acid, nitric acid, sulfuric acid, phosphoric acid, and organic acids such as formic acid, lactic acid, citric acid, oxalic acid, methane sulfonic acid, benzene sulfonic acid, benzoic acid, acetic acid, trifluoracetic acid, propionic acid, and fumaric acid.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

The present invention relates to the process of manufacturing IF7-SN38. The process involves three steps as described in FIGS. 1-3: (1) Synthesis of BCH; (2) Synthesis of BCH-SN38; (3) Synthesis of IF7-SN38. Unlike the method described in the prior art publication, the present invention describes a process that is simple, scalable and economical, and utilizes highly pure BCH-SN38 to produce IF7-SN38 in high yield and purity without a large-scale reverse phase column chromatography purification process.

When IF7-SN38 was injected intravenously into nude mice carrying human colon HCT116 tumors, it efficiently suppressed tumor growth at low dosages with no apparent side effects. These results indicated that IF7 peptide serves as an efficient drug delivery vehicle by targeting Anxal expressed on the surface of tumor vasculature.

It is an object of the present invention to provide a tumor-vascular targeting antitumor agent.

It is another object of the present invention to provide a tumor-vascular targeting antitumor agent wherein the antitumor agent comprises an annexin-1 binding peptide conjugated to an anticancer drug through a linker.

It is yet another object of the present invention to provide a tumor-vascular targeting antitumor agent wherein the annexin-1 binding peptide is a peptide having the sequence IFLLWQR (IF7).

It is still another object of the present invention to provide a tumor-vascular targeting antitumor agent wherein the anticancer drug is 7-Ethyl-10-hydroxycamptothecin (SN38).

It is another object of the present invention to provide a tumor-vascular targeting antitumor agent wherein the linker is 4-{4-[(N-maleimydomethyl)cyclohexanecarboxamido]methyl} cyclohexane-1-carboxylic acid (BCH).

It is a further object of the present invention to provide a process of manufacturing IF7-SN38.

It is yet another object of the present invention to provide a process of manufacturing IF7-SN38 wherein the process comprises:

a) synthesizing BCH;
b) synthesizing BCH-SN38; and
c) synthesizing IF7-SN38

### BRIEF DESCRIPTION OF THE DRAWINGS

The novel features of this invention, as well as the invention itself, both as to its structure and its operation, will be best understood from the accompanying drawings, taken in conjunction with the accompanying description, and in which:

FIG. 1 illustrates the chemical synthesis of BCH.

FIG. 2 illustrates the chemical synthesis of BCH-SN38.

FIG. 3 illustrates the chemical synthesis of IF7-SN38.

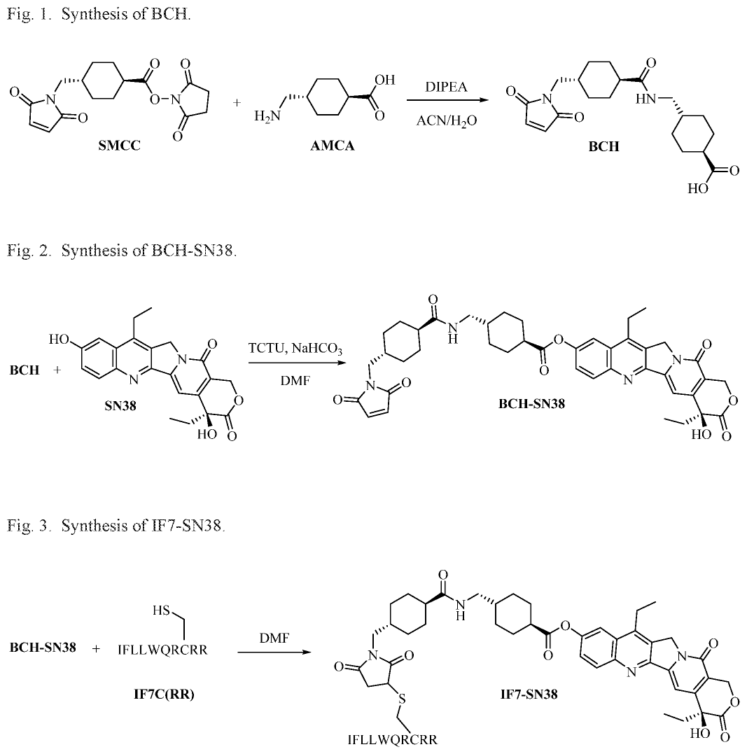

The invention can be better visualized by turning now to the following examples.

### DETAILED DESCRIPTION OF THE INVENTION

### Acronyms for the Chemicals and Reagents

SMCC Succinimidyl 4-(N-maleimidomethyl)cyclohexane-1-carboxylate
AMCA Trans-4-(Aminomethyl)cyclohexanecarboxylic acid (Tranexamic acid)
BCH 4-{4-[(N-maleimydomethyl)cyclohexanecarboxamido]methyl} cyclohexane-1-carboxylic acid
SN38 7-Ethyl-10-hydroxycamptothecin
IF7C(RR) IFLLWQR-C-RR peptide
TCTU O-(6-chlorobenzotriazol-1-yl)-N,N,N′,N′-tetramethyluronium tetrafluoroborate
DIPEA Diisopropylethylamine
ACN Acetonitrile
DMF Dimethylformamide
MTBE Methyl tert-butyl ether
DCM Dichloromethane

IF7-SN38 is a novel anti-cancer pro-drug for targeted therapy. A competitor drug that is currently in clinical trials, is polyethylene-glycol conjugated SN38 micelle (PEGylated SN38), which penetrates tumors through disorganized endothelial cell layers and claimed to be effective against brain tumor cells. The clinical trials for this drug have been sponsored by US companies, NEKTOR and Enzon. The Phase II clinical trials by Enzon on colorectal cancers was unsuccessful. On the other hand, NEKTOR's sponsored clinical trial in Phase II on brain tumors has been successful and the drug has been moved to Phase III.

PEG-SN38 overcomes blood brain barrier, but does not target brain tumors. As a result, patients are injected with a high dosage of PEGylated SN38, which causing serious side effects. By contrast, due to the unique property of the IF7 peptide, IF7-SN38 targets brain tumors with high efficiency and overcomes blood brain barrier by passive transcytosis mechanism. Therefore, using our targeted therapy, a small amount of IF7-SN38 is required to be injected to patients. The efficiency of IF7-SN38 is unprecedented in mice, in particular for the brain malignancies. Experimental studies indicated that treatment of mice with tumors using IF7-SN38 resulted in significant shrinkage in tumors size without producing any side effects in the treated mice. It is believed that IF7-SN38 would work far better than PEGylated SN38 by targeting malignant tumors including brain malignancy. The low dosage, specificity, and easily degrading nature of IF7-SN38 in targeted cells to release SN38, are the major factors for the superiority of our drug to PEG-SN38.

The current invention provides a reliable reproducible method for manufacturing large quantity of the API for the clinical studies.

### Examples

The previous milligram scale preparation of IF7-SN38 lacked the required properties and process characteristics for large scale production of the API. Significant process development and optimization were conducted to establish a robust and reproducible process to overcome the problems for our imminent needs for large quantities of the API for the next phases of the campaign beyond the in vitro and in vivo studies. The present invention, provide an efficient, robust, and cost effective process for the large scale manufacturing of IF7-SN38 in high yield and purity.

An analytical reverse-phase, high performance liquid chromatography (HPLC) system was used to develop a HPLC method suitable for detecting all components of the 3-Step process. The HPLC method was finally optimized and used for monitoring all reactions. The area percent purity of the products and the rate of the reactions were monitored and assessed under different reaction conditions. The best conditions were selected based on the overall purity profile of the reaction mixtures and the compatibility of the reaction conditions for large scale manufacturing of the API under cGMP conditions. All critical process parameters (CPPs), including type and stoichiometry of the reactants and reagents, order of addition of the reagents, temperature, time, type of reaction solvents, and alternative work up (acidic, neutral, and basic) of the reaction mixtures were evaluated in the development process and optimal reaction conditions were selected for the process.

A representative procedure for the preparation of approximately 10 grams of a Proof-of-Concept batch of IF7-SN38 is provided in detail below, but the batch size may be increased or decreased as needed. It is further emphasized that the temperature ranges, weight and volumes for the reagents and solvents, and the reaction times are exemplary for said batch size, and should not be construed as being limiting. These parameters may be varied depending on the batch size desired. It is well understood in the art that minor deviations from the specified procedure do occur occasionally and are permissible within the scope of the invention. The methods of the present invention are detailed in the following procedures which are offered by way of illustration and are not intended to limit the scope of the invention in any manner.

The first step of the process is the synthesis of BCH under the optimized conditions as described below.

### Step 1

| Reagent | MW | Amount | | mmol | Equiv. |
|---|---|---|---|---|---|
| SMCC | 334.32 | 4.07 | g | 12.174 | 1 |
| AMCA | 157.21 | 2.39 | g | 15.217 | 1.25 |
| Acetonitrile | | 40.7 | mL | | 10 vol. |
| DI Water | | 20.3 | mL | | 5 vol. |
| DIPEA | 129.25 | 0.526 | mL | 3.043 | 0.25 |

Purge a 100-mL, three-neck cylindrical flask, equipped with a mechanical stirrer, a J-Kem temperature controller, and a nitrogen inlet, with nitrogen
Charge the flask with SMCC and AMCA, followed by acetonitrile and water with stirring
Add DIPEA slowly and allow the mixture stir at ambient temperature (22±2° C.) overnight (14 h)
Analyze the mixture by HPLC for disappearance of SMCC and formation of BCH
Dilute the reaction mixture with MTBE (61 mL) and stir for 5 minutes
Filter the solid and wash with MTBE (2×20 mL)
Dissolve the resulting white solid in 20% MeOH/DCM (407 mL)
Wash the organic solution with 15% brine solution (2×40.7 mL)
Separate the organic solution and extract the combined aqueous solution with DCM (40.7 mL)
Dry the combined organic solution over Na₂SO₄ (270 g) for 10 min
Filter the solution and wash the filter with DCM (122 mL)
Concentrate the solution to a white solid at 20-25° C.
Slurry the solid in acetone (61 mL) at ambient temperature for 30 min
Filter the solid and wash with acetone (20 mL) and 1:1 MTBE/acetone (2×20 mL)
Dry the solid under high vacuum at 20-30° C. for a minimum of 24 h

This process gave BCH as a white solid (3.106 g, 68% yield) with a purity of >99.9% by HPLC and NMR. Analytical data including, ¹H-NMR & ¹³C-NMR, and Mass spec were consistent with the structure of the molecule.

The second step of the process is the synthesis of BCH-SN38 under the optimized conditions as described below.

### Step 2

| Reagent | MW | Amount | mmol | Equiv. |
|---|---|---|---|---|
| SN38 | 392.40 | 2.909 g | 7.414 | 1 |
| BCH | 376.45 | 3.070 g | 8.155 | 1.1 |
| TCTU | 355.53 | 3.031 g | 8.526 | 1.15 |
| NaHCO₃ | 84.01 | 2.180 g | 25.949 | 3.5 |
| Na₂SO₄ | 142.04 | 2.909 g | 20.480 | 2.8 |
| DMF | | 58 mL | | 20 vol. |

Purge a 500-mL, three-neck cylindrical flask, equipped with a mechanical stirrer, a J-Kem temperature controller, and a nitrogen inlet, with nitrogen
Charge the flask with BCH, SN38, TCTU and Na₂SO₄
Add anhydrous DMF at (0±1° C.) with stirring
Add NaHCO₃ in one portion and allow the mixture to stir at the same temperature
Analyze the batch by HPLC after 3 h
Quench the batch after 4 h
Cool the batch to −5±5° C.
Dilute the batch with a cold aqueous solution of 0.08 M HCl (290 mL) in one portion with stirring
Stir the batch for 5 min
Filter the batch and wash with ice-cold solution of 20% DMF/water (3×60 mL) and MTBE (2×60 mL)
Dissolve the wet solid in DCM (290 mL) and wash with 0.01 M HCl (120 mL) and brine (120 mL)
Dry the organic solution over sodium sulfate for 10 min
Filter and wash the filter with DCM (2×120 mL)
Concentrate the solution and re-dissolve the solid in DCM (60 mL)
Dilute the DCM solution with MTBE (350 mL) with stirring
Filter the solid and wash the filter-cake with 6:1 MTBE/DCM (60 mL) and MTBE (60 mL)
Dry the solid under high vacuum at 25-30° C. for a minimum of 18 h

This process yielded BCH-SN38 as pale yellow solid (4.98 g, 89% yield) with a purity of 97.7% by HPLC and NMR. Analytical data including, ¹H-NMR & ¹³C-NMR, and Mass spec were consistent with the structure of the molecule.

The final step of the process is the synthesis of IF7-SN38 under the optimized conditions as described below.

### Step 3

| Reagent | MW | Amount | mmol | Equiv. |
|---|---|---|---|---|
| BCH-SN38 | 750.85 | 4.00 g | 5.327 | 1 |
| IF7C(RR) | 1390.73 | 9.70 g | 6.978 | 1.31 |
| DMF | | 60 mL | | 15 vol. |

Purge a 500-mL, three-neck cylindrical flask, equipped with a mechanical stirrer, a J-Kem temperature controller, and a nitrogen inlet, with nitrogen
Charge the flask with BCH-SN38
Add anhydrous DMF (12 mL) at ambient temperature (22±2° C.) with stirring
Add a solution of IF7C(RR) in anhydrous DMF (48 mL) slowly with stirring.
Allow the mixture to stir at the same temperature overnight
Analyze the batch by HPLC for complete reaction after 15 h
Quench the batch after 16 h
Dilute the batch with acetonitrile (400 mL) with stirring
Stir the batch for 10 min
Filter the solid and wash the filter-cake with acetonitrile (3×80 mL)
Slurry the wet solid in acetonitrile (80 mL) for 1 h
Filter the solid and wash the filter-cake with acetonitrile (2×40 mL)
Repeat the acetonitrile slurry process one more time
Filter the solid and wash the filter-cake with acetonitrile (2×40 mL)
Dry the solid under high vacuum at 25-30° C. for a minimum of 3 days

This process yielded IF7-SN38 as pale yellow solid (11.374 g, 90% yield) with a purity of 96.8% by HPLC and NMR as its TFA salt. TFA is the counter ion of the IF7 peptide and will be transferred to the API in the final step. Analytical data including, ¹H-NMR & ¹³C-NMR, and Mass spec were consistent with the structure of the product.

Syntheses of other salt forms of IF7-51\138, particularly the HCl salt, will be achieved using the corresponding salts of the peptide. List the corresponding salts

One embodiment of the present invention provides a tumor-vascular targeting antitumor agent.

Another embodiment of the present invention provides a tumor-vascular targeting antitumor agent wherein the antitumor agent comprises an annexin-1 binding peptide conjugated to an anticancer drug through a linker.

Yet another embodiment of the present invention provides a tumor-vascular targeting antitumor agent wherein the annexin-1 binding peptide having the peptide sequence IFLLWQR (IF7).

A further embodiment of the present invention provides a tumor-vascular targeting antitumor agent wherein the anticancer drug is 7-Ethyl-10-hydroxycamptothecin (SN38).

Another embodiment of the present invention provides a tumor-vascular targeting antitumor agent wherein the linker is 4-{4-[(N-maleimydomethyl)cyclohexanecarboxamido]methyl} cyclohexane-1-carboxylic acid.

A further embodiment of the present invention provides a process of manufacturing IF7-SN38.

Still another embodiment of the present invention provides a process of manufacturing IF7-SN28 wherein the process comprises:

a) synthesizing BCH;
b) synthesizing BCH-SN38; and
c) synthesizing IF7-SN38.

Yet another embodiment of the present invention provides a process for manufacturing an anti-cancer compound capable of targeting a tumor, the anti-cancer compound having a final structure of Formula I,

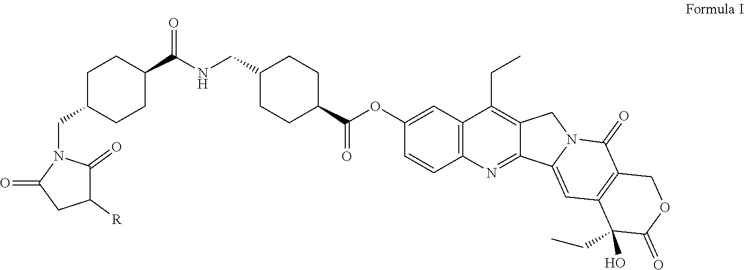

wherein R is a peptide comprising an amino acid sequence of IFLLWQRX₁X₂X₃,
(a) providing a linker having the final structure of:

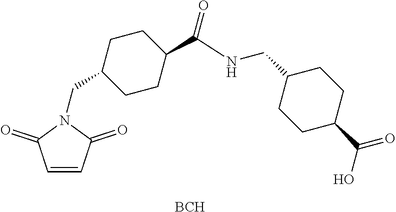

wherein the linker is formed by coupling succinimidyl 4-(N-maleimidomethyl)cyclohexane-1-carboxylate (SMCC) and trans-4-(Aminomethyl)cyclohexanecarboxylic acid (AMCA), according to the following synthesis:

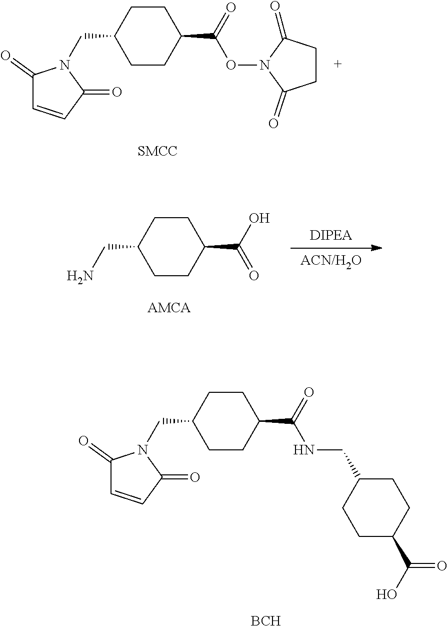

(b) providing a moiety for attachment to the linker, wherein the moiety is a camptothecin analog, further wherein the moiety is attached to the linker, resulting in a moiety-linker product; and
(c) conjugating the moiety-linker product of (b) with R in order to arrive at Formula I.

Still another embodiment of the present invention provides a process of manufacturing an anti-cancer compound capable of targeting a tumor wherein the process further comprises:
(d) purifying the linker;
(e) purifying the linker-moiety product; and
(f) purifying the product of Formula I.

A further embodiment of the present invention provides a process of manufacturing an anti-cancer compound capable of targeting a tumor wherein a base and at least two solvents are employed, the base comprising diisopropylethylamine and the solvents comprising acetonitrile and water.

Another embodiment of the present invention provides a process of manufacturing an anti-cancer compound capable of targeting a tumor wherein the BCH linker is purified by slurry/trituration. In a preferred embodiment the linker-moiety product is purified by slurry/trituration. In another preferred embodiment the anti-cancer compound is purified by slurry/trituration. In still another preferred embodiment at least one solvent is employed in the slurry/trituration, the at least one solvent comprising acetonitrile.

Yet another embodiment of the present invention provides a process of manufacturing an anti-cancer compound capable of targeting a tumor wherein at least two solvents are employed in the slurry/trituration, the at least two solvent comprising acetone and methyl tert-butyl ether.

Still another embodiment of the present invention provides a process of manufacturing an anti-cancer compound capable of targeting a tumor wherein the linker-moiety product is prepared by coupling of the linker and the camptothecin analog.

A further still embodiment of the present invention provides a process of manufacturing an anti-cancer compound capable of targeting a tumor wherein the coupling takes place in the presence of O-(6-chlorobenzotriazol-1-yl)-N,N,N′,N′-tetramethyluronium tetrafluoroborate and sodium or potassium sulfate.

Another embodiment of the present invention provides a process of manufacturing an anti-cancer compound capable of targeting a tumor wherein a base and at least one solvent are employed, wherein the base is selected from the group consisting of sodium and potassium hydrogen carbonate and the solvent is dimethylformamide or equivalent.

Yet another embodiment of the present invention provides a process of manufacturing an anti-cancer compound capable of targeting a tumor wherein the anti-cancer compound having the structure of Formula I is prepared by coupling of the linker-moiety product and the peptide of R. In a preferred embodiment the coupling takes place in dimethylformamide or equivalent.

A further embodiment of the present invention provides a process of manufacturing an anti-cancer compound capable of targeting a tumor wherein the linker-moiety product is conjugated to R at the X1 position.

Still another embodiment of the present invention provides a process of manufacturing an anti-cancer compound capable of targeting a tumor wherein X₂ and X₃ are the same amino acid. In another embodiment, X₂ and X₃ are different amino acids.

It will be appreciated that details of the foregoing embodiments, given for purposes of illustration, are not to be construed as limiting the scope of this invention. Although several embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

What is claimed is:

1. A process for manufacturing an anti-cancer compound capable of targeting a tumor, the anti-cancer compound having a final structure of Formula I, 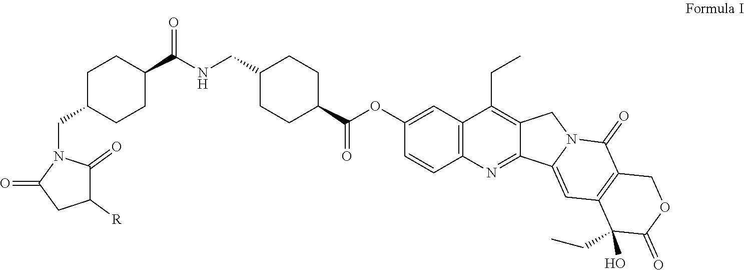
wherein R is a peptide comprising an amino acid sequence of IFLLWQRX₁X₂X₃, the process comprising the steps of:
(a) providing a linker having a final structure of: 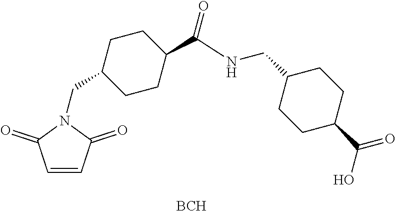
wherein the linker is formed by coupling succinimidyl 4-(N-maleimidomethyl)cyclohexane-1-carboxylate (SMCC) and trans-4-(Aminomethyl)cyclohexanecarboxylic acid (AMCA), according to the following synthesis: 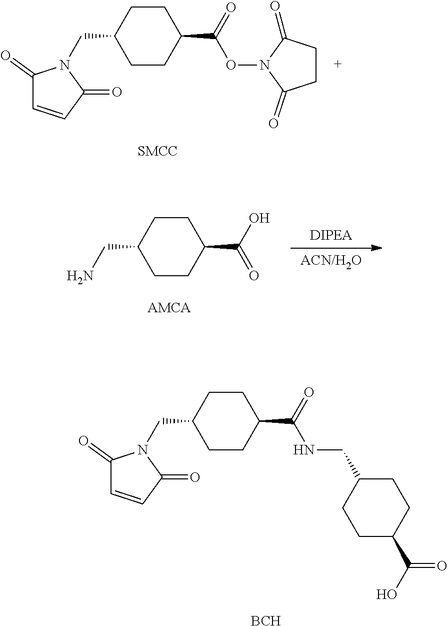
(b) providing a moiety for attachment to the linker, wherein the moiety is a camptothecin analog, further wherein the moiety is attached to the linker, resulting in a moiety-linker product; and
(c) conjugating the moiety-linker product of (b) with X in order to arrive at Formula I.

2. The process of claim 1, further comprising:
(d) purifying the linker;
(e) purifying the linker-moiety product; and
(f) purifying the product of Formula I.

3. The process of claim 2, wherein a base and at least two solvents are employed, the base comprising diisopropylethylamine and the solvents comprising acetonitrile and water.

4. The process of claim 1, wherein the linker is purified by slurry/trituration.

5. The process of claim 4, wherein at least two solvents are employed in the slurry/trituration, the at least two solvent comprising acetone and methyl tert-butyl ether.

6. The process of claim 1, wherein the linker-moiety product is prepared by coupling of the linker and the camptothecin analog.

7. The process of claim 6, wherein the coupling takes place in the presence of O-(6-chlorobenzotriazol-1-yl)-N,N,N′,N′-tetramethyluronium tetrafluoroborate and sodium or potassium sulfate.

8. The process of claim 6, wherein a base and at least one solvent are employed, wherein the base is selected from the group consisting of sodium and potassium hydrogen carbonate and the solvent is dimethylformamide or equivalent.

9. The process of claim 1, wherein the linker-moiety product is purified by slurry/trituration.

10. The process of claim 9, wherein at least two solvents are employed in the slurry/trituration, the at least two solvents comprising dichloromethane and methyl tert-butyl ether.

11. The process of claim 1, wherein the anti-cancer compound having the structure of Formula I is prepared by coupling of the linker-moiety product and the peptide of R.

12. The process of claim 11, wherein the coupling takes place in dimethylformamide or equivalent.

13. The process of claim 1, wherein the anti-cancer compound is purified by slurry/trituration.

14. The process of claim 13, wherein at least one solvent is employed in the slurry/trituration, the at least one solvent comprising acetonitrile.

15. The process of claim 1, wherein the linker-moiety product is conjugated to R at the X₁ position.

16. The process of claim 1, wherein X₂ and X₃ are the same amino acid.

17. The process of claim 1, wherein X₂ and X₃ are different amino acids.
